(19) United States
(12) Patent Application Publication
(10) Pub. No.: US 2023/0047105 A1
(43) Pub. Date: Feb. 16, 2023

(54) LEFT CORONARY ARTERY STENT SYSTEM, STENT ASSEMBLY AND METHOD THEREFORE

(71) Applicant: Douglas A. Tomasian, Lake Barrington, IL (US)

(72) Inventor: Douglas A. Tomasian, Lake Barrington, IL (US)

(21) Appl. No.: 17/400,673

(22) Filed: Aug. 12, 2021

(51) Int. Cl. A61F 2/958

(52) U.S. Cl. CPC A61F 2/958; A61F 2/954

(57) ABSTRACT

A left coronary artery (“LCA”) stent system includes: a first guidewire having a first diameter; a second guidewire having a second diameter, which may be the same or different than the first diameter; a left main (“LM”) stent assembly including (i) a catheter including a saline/contrast solution lumen and a guidewire lumen sized to accept both the first and second guidewire diameters of the first and second guidewires, and (ii) an LM stent sized to be deployed within a patient's LM; a left anterior descending artery (“LAD”) stent assembly for insertion through a deployed LM stent and along the first guidewire; and a circumflex artery (“LCx”) stent assembly for insertion through the deployed LM stent and along the second guidewire.

### BACKGROUND

The present disclosure relates generally to the treatment of cardiovascular disease and in particular to the use of cardiovascular stents for left coronary artery disease.

The left main coronary artery typically bifurcates or trifurcates shortly after its origin. FIG. 1 illustrates that in most instances, the left coronary artery (“LCA”) has a left main (“LM”) that bifurcates into two branches, namely, the left anterior descending artery (“LAD”) and the circumflex artery (“LCx”). The anatomy of the LM, the LAD and the LCx is problematic for treatment via stents because the LM typically has a large diameter, around 4 mm conventionally, and because there is a significant drop-off in the diameters of the LAD and the LCx from the LM, which are typically 3.5 mm in diameter or smaller. In the prior art method shown below, a single stent has to cover the LM and one of the branches (e.g., the LAD), and therefore has to accommodate the drop-off in diameter.

Another problem is that disease is typically present in both branches, the LAD and LCx. LCA disease is placed into seven categories, namely, (i) 1, 1, 1—disease present in all three of LM, LAD and LCx, (ii) 0, 1, 1—disease present in LAD and LCx branches only, (iii) 1, 1, 0—disease present in LM and LAD only, (iv) 1, 0, 1—disease present in LM and LCx only, (v) 1, 0, 0—disease present in LM only, (vi) 0, 1, 0—disease present in LAD only, and (vii) 0, 0, 1—disease present in LCx only. The most common LCA disease involves disease in both the LAD and LCx branches, namely, (i) and (ii) above.

The following figures illustrate a known double kiss crush (“DKcrush”) technique for treating 1, 1, 1 LCA disease with a stent. FIG. 2 illustrates the LM branching into the LAD and the LCx, each of which are diseased with plaque build-up. In FIG. 3, guidewires are inserted in a first step of the DKcrush technique through the LM, one each into the LAD and LCx. In a second step (not illustrated), the LM and LAD are pretreated with a balloon introduced along the LM and LAD guidewire to compress the plaque against the wall of the vessels. In a third step (not illustrated), the LCx is pretreated with a balloon introduced along the LCx guidewire to compress the plaque against the wall of that vessel.

FIG. 4 illustrates a fourth step in which a first stent is advanced along the LCx guidewire into the LCx. The longer-term durability of the DKcrush technique is usually hampered most by the LCx, which is why the first stent is typically deployed in the LCx. Prior single stent deployments across the ostium of the LCx without side branch stenting of the LCx, (usually side branch ballooning only) were likewise hampered with ostial LCx renarrowing, which in part led to the development of more complicated crush stent techniques, such as the DKcrush, to attempt to cover all of the vessel treated, including the LCx.

FIG. 5 illustrates a fifth step in which a balloon is advanced along the LM and LAD guidewire into the LM and LAD. FIG. 6 illustrates a sixth step in which a balloon within the LCx stent is inflated to deploy the LCx stent. FIG. 7 illustrates a seventh step in which the catheter and balloon are removed from the deployed LCx stent along the LCx guidewire, leaving only the deployed LCx stent, the LCx guidewire and the LM/LAD balloon. Notably, the upper, proximal corner of the deployed LCx stent extends into the passageway where the LM transitions to the LAD.

FIG. 8 illustrates an eighth step in which the balloon introduced in FIG. 5 into the LM and LAD is inflated to crush the upper, proximal corner of the deployed LCx stent, such that the corner is bent downwardly so as to be at least substantially flush with the opening into the LCx. In a ninth step (not illustrated), (i) the balloon just inflated is removed from the LM and LAD along the LM and LAD guidewire and (ii) the LCx guidewire is rewired through the crushed struts of the LCx stent.

FIG. 9 illustrates a tenth step in which two new balloons are inserted, namely, a first balloon is inserted along the LCx guidewire and though the crushed struts of the LCx stent, while a second balloon is inserted along the LM and LAD guidewire into the LM and LAD. FIG. 10 illustrates an eleventh step in which the two new balloons are inflated, such that the two balloons touch at their proximal ends, performing the “first kiss”. The dual inflations of the first kiss serve to open the LCx stent strut through which the LCx balloon has been inserted (to provide an opening for blood flow), while the LM and LAD balloon maintains the crushed upper, proximal corner of the deployed LCx stent in a flattened or flush state.

In a twelfth step (not illustrated), the two balloons are removed from the LCx and the LM/LAD. The guidewire is also removed from the LCx, leaving only the crushed LCx stent and the LM and LAD guidewire. FIG. 11 illustrates a thirteenth step in which a second stent is advanced along the LM and LAD guidewire into the LM and LAD. FIG. 12 illustrates a fourteenth step in which a balloon within the LM and LAD stent is inflated to deploy the LM and LAD stent.

FIG. 13 illustrates a fifteenth step in which the catheter and balloon are removed from the deployed LM and LAD stent along the LM and LAD guidewire, leaving only the deployed LCx and LM/LAD stents and their respective guidewires. Additionally, the LCx guidewire is again rewired to now extend through two layers of struts (LM/LAD stent and crushed LCx stent struts).

FIG. 14 illustrates a sixteenth step in which two additional new balloons are inserted, namely, a first additional balloon is inserted along the LCx guidewire and the through the struts of the LM and LAD stent and the crushed struts of the LCx stent, while a second additional balloon is inserted along the LM and LAD guidewire into the LM and LAD. FIG. 15 illustrates a seventeenth step in which the two additional new balloons are inflated, such that the two additional balloons touch at their proximal ends, performing the “second kiss”. The dual inflations of the second kiss serve the same purpose as those of the first kiss, except that here, the LCx balloon extends through two layers of metal struts. The LCx balloon opens the LCx stent strut and the LM/LAD stent strut through which the LCx balloon has also been inserted (to provide and maintain an opening for blood flow), while the LM and LAD balloon maintains the crushed upper, proximal corner of the deployed LCx stent in a flattened or flush state.

In an eighteenth and final step (not illustrated), the catheters, balloons and guidewires are removed, leaving the deployed LCx and LM/LAD stents.

A number of problems are caused by the above-described technique. First, the technique crushes and mangles the LCx stent a manner for which it has not been designed. A stent is in general a metal tube, which is cylindrical and designed to be implanted into a cylindrical artery. Stents are not designed to provide side branch access or to have holes punched through their struts. Stents are designed to have radial strength, and their strut structure is generally small, so when the technique forces insertion through one stent, into another stent, after which a balloon is inflated through two layers of metal, the risk of a thicker thrombus or clotting is created, which limits the ability of a drug from a drug-eluting stent, if used, to reach a corresponding vessel wall.

The above technique also creates procedural hazards, including the physician having to reach the correct location in the middle portion of the LM and LAD stent to access the crushed end of the LCx stent (see FIG. 13). If the correct location is not reached, and the reinserted LCx guidewire extends underneath the crushed portion of the LCx strut instead of through the crushed portion, the LCx stent will likely become mangled in an undesirable way when the balloon is inflated. Namely, the LCx stent may become renarrowed, misshapen and moved away from the vessel walls, creating turbulent flow and possibly attracting thrombus, as metal in the lumen of a small vessel will often attract thrombus. A stent deployed in a vessel without being placed fully into the walls of the vessel is known as an incomplete stent apposition, and is associated with poor outcomes such as increased cardiac events in the stented vessel. Stent apposition occurs even in procedures without crush techniques in which the stent architecture is not altered. When a stent apposition occurs, rework is needed, and the patient may become unstable as flow through the entire left main stent system can become compromised, potentially causing anxious moments for the physician while the patient is on the operating table.

Additionally, rewiring into the correct lumen of the stent is necessary in the known technique but may be very technically challenging. The patient may also become unstable if flow in the LCx is compromised due to the stent becoming misshapen during the prior inflation. Complicating the above-described situation is the fact that patients may become unstable during stenting procedures even when the LCx is ballooned in an appropriate fashion. Determining what the actual procedure will entail may also be challenging, as angiography alone provides a two-dimensional image of the vessel and may not clearly show the relationship of the stent to the vessel walls. Higher level imaging such as intravascular ultrasound, which requires the advancement of an ultrasound catheter over the guidewire, and which can acquire images of the vessel wall and stent from the inside of the vessel, may be used to assist the physician, however, such imaging can be challenging as delivering an intravascular ultrasound catheter through stent struts can be difficult. Here, the patient may become more unstable if blood flow becomes further compromised when the catheter is introduced, consuming the lumen space that allows blood flow.

To summarize, problems with the DKcrush technique method involve (i) inappropriate stent function over the long term because stents are not designed to be used in the manner described above, (ii) increased thrombus, (iii) renarrowing at the stent and associated rework, and (iv) as mentioned at the top, a size mismatch between the LM and LAD, which the DKcrush technique attempts to address using a single stent constructed to have a uniform deployed diameter.

There are other techniques besides the DKcrush for stenting LCA disease. A Minicrush technique exists that reduces the number of steps described above for the Dkcrush. The Minicrush technique however still requires that a balloon be inserted through a strut in the middle portion of the LM stent and another strut in the crushed end of the LCx stent, similar to FIG. 14 above. Problems (i) to (iv) described above for the DKcrush technique accordingly still persist for the Minicrush technique.

FIG. 16 illustrates that a Culotte technique also exists, which involves the deployment initially of an LCx stent having a proximal end that extends into the LM and a distal end that extends into the LCx. A guidewire is then extended though a strut of the LCx stent into the LAD, after which an LAD balloon is introduced along the guidewire and through the strut, such that a proximal end of the balloon remains in the LM and a distal end of the balloon extends into the LAD. The balloon is then inflated to open a large hole in the LCx stent, splitting apart multiple struts. An LAD stent is then introduced along the guidewire and through the large hole, such that a proximal end of the LAD stent remains within the proximal end of the LCx stent in the LM and a distal end of the LAD stent extends into the LAD. The LAD stent is then ballooned into deployment having a proximal portion residing within the proximal end of the LCx stent and a distal portion deployed in the LAD.

Problems also exist with the Culotte technique. For example, the large hole formed in the LCx stent materially degrades its function over the long term.

An additional technique is used when the hemodynamic status of the patient is unstable. FIG. 17 illustrates a deployment for an unstable patient, wherein the proximal ends of the LAD stent and the LCx stent reside side-by-side in the LM. An obvious hazard here is the proximal end of one of the LAD and LCx stents occluding the other. The technique of FIG. 17 is relatively simple to deploy and used for temporary situations, for example, to prevent a patient on the verge of cardiac arrest from undergoing same. The technique of FIG. 17 is accordingly not as relevant to the permanent LM stent deployment of the present disclosure. The technique of FIG. 17 may also not be optimal because the two stents creating two pathways inside the LM can lead to thrombus. Also, re-access to the LAD or LCx may be difficult. Further, the stent sizes may collectively form a non-circular cross-section that does not properly cover the inner wall of the LM.

Given the above-described difficulties in treating LCA disease using conventional techniques, and the fact that the LCA supplies blood to, in many patients, two-thirds (and in some cases even more) of the myocardial muscle mass conventionally, and the risk involved with potentially occluding blood to this much of the heart even for a short period of time without reserve (e.g., without the patient being connected to a heart lung machine), treatment of LCA disease is typically left within the realm of bypass surgery. There are however obvious benefits to treating cardiovascular disease via stent and without bypass if performed safely.

A need exists accordingly for an improved apparatus and associated methodology for treatment of left coronary artery cardiovascular disease using cardiovascular stents.

### SUMMARY

The examples described herein disclose a left coronary artery (“LCA”) stent system, assembly and associated method of deploying same. The LCA system includes a first stent that when deployed resides in the short but wider left main (“LM”), and which is not intended to extend into either the left anterior descending artery (“LAD”) or the circumflex artery (“LCx”). The LM stent is relativelty short in length, e.g., 6 mm to 24 mm long, such that the LM stent does not extend significantly into the LAD or LCx or extend in the other direction into the aorta. The LM stent is also relatively wide in diameter, e.g., 4 mm to 6 mm in diameter, depending on the diameter of the patient's LM.

The relatively large diameter of the LM stent is advantageous to the overall system because the ballon catheter of the stent is correspondingly relatively large, allowing enough room for (i) a saline/contrast solution lumen or pathway to be provided inside the catheter for inflating the balloon, and (ii) a guidewire lumen to be provided that allows two guidewires to be exended through an interior of the catheter. In one embodiment, the two guidewires are 14/1000 inch in diameter, which is 0.36 mm. The diameters of the guidewires may be the same or different. Two of such wires placed side-by-side may therefore span a collective distance of about 0.72 mm. The guidewire lumen may be non-circuar and have a longest dimention of at least 0.72 mm and a perpendicular width of at least 0.36 mm, which fit within a 4 French (“Fr”) catheter that typically has an outer diameter of 1.35 mm. As illustrated below, the 4 Fr catheter provides plenty of cross-sectional wall space within which to co-extrude the saline/contrast solution lumen along with the above-dimensioned guidewire lumen.

The saline/contrast solution lumen may be small because it only needs to carry a small amount of saline/contrast solution to the ballon for inflation. In an embodiment illustrated herein, the saline/contrast solution lumen may be cresent or half-moon shaped to efficiently use space within the catheter wall adjacent to the guidewire lumen. Thinner guidewires may be used to allow for thinner ballon catheters and different catheter materials may require different wall thicknesses. The above numbers are therefore meant to provide one working example. In other examples, a 5 Fr (1.67 mm outside diamenter) to an 8 Fr (2.7 mm outisde diameter) LM catheter may be provided to accept larger diameter guidewires.

Two guidewires may be used when the patient has disease in both the LAD and the LCx, which is the most common scenario. However, if the patient has disease in only one of the LAD or LCx, then only one guidewire is needed. Or, a single guidewire may be rewired to carry different branch stents at different times.

Once the LM stent is deployed, the LM catheter is removed, leaving a large, stented access for both the LAD and the LCx, where guidewires run to both vessels (assuming both are diseased). LAD and LCx stents are sized respectively for the patient's LAD and LCx and are inserted through the stented LM access and deployed, one after the other, and in an order desired by the physician. In an alternative embodiment, LAD and LCx stents may be deployed simultaeously. In any case, the LAD and LCx catheters and associated guidewires are then removed. A final angiogram is performed to ensure a good result. An advantage of the present method is that the entire vessel is covered with stent, but no segment of the stent is “crushed” or malformed, allowing the stents of the LM as well as the LAD and LCx to maintain their structural integrity/radial force.

It is contemplated to provide at least one balloon inflation after the guidewires are initially installed to pretreat the LM, LAD and LCx as needed before the LM stent is deployed. For example, the LM and LAD may be treated initially by a first balloon inserted along the LM and LAD guidewire. The LM and LAD balloon and associated catheter are then removed. The LCx is then treated by a second balloon inserted along the LCx guidewire. The LCx balloon and associated catheter are then removed. The LM stent may then be deployed as described above.

If the LM stent is not short enough to fit completely within the LM, it may be possible to let a small portion of the stent not adhering to any vessel wall to reside at a location just downstream of where the LM branches into the LAD and LCx, forming a stent septum of metal. It is believed that forming a short unnattached stent septum is much preferable to the prior art methods described above. Also, while two guidewires are inserted in one embodiment, it is also contemplated to insert an initial guidewire through the LM and into one of the LAD or LCx, deploy the LM stent, and then (i) insert a second guidewire through the large, stented LM access and into the other of the LAD or LCx or (ii) rewire the first guidewire through the large, stented LM access and into the other of the LAD or LCx.

Similarly, if there are three branches off of the patent's LM as is sometimes the case, it is conteplated to initially insert two guidewires into two of the branches, deploy the LM stent, remove the LM stent catheter, deploy a first branch stent along a first one of the guidewires and through the large, stented LM access into a first branch, remove the first branch stent catheter, deploy a second branch stent along a second one of the guidewires through the large, stented LM access into a second branch, remove the second branch stent catheter, remove the two guidewires, insert a third guidewire into the third branch, deploy a third branch stent along the third guidewire and through the large, stented LM access into the third branch, and remove the third branch stent catheter and the third guidewire. In a first alternative embodiment, one of the first and second guidewires is rewired into the third branch instead of using a third guidewire. In a second alternative embodiment, a single guidewire is used initially for the first branch and then rewired into the second and third branches.

In light of the disclosure herein and without limiting the disclosure in any way, in a first aspect of the present disclosure, which may be combined with any other aspect, or portion thereof, a left coronary artery (“LCA”) stent system includes: a first guidewire having a first diameter; a second guidewire having a second diameter, which may be the same or different than the first diameter; a left main (“LM”) stent assembly including (i) a catheter having a saline/contrast solution lumen and a guidewire lumen, the guidewire lumen sized to accept both the first and second guidewire diameters of the first and second guidewires, and (ii) an LM stent sized to be deployed within a patient's LM; a left anterior descending artery (“LAD”) stent assembly for insertion through a deployed LM stent and along the first guidewire; and a circumflex artery (“LCx”) stent assembly for insertion through the deployed LM stent and along the second guidewire.

In a second aspect of the present disclosure, which may be combined with any other aspect, or portion thereof, the LM stent is sized to be deployed within the patient's LM only.

In a third aspect of the present disclosure, which may be combined with any other aspect, or portion thereof, the LM stent is sized to be deployed within the patient's LM and at least a portion of a transition section in which the LM branches into the LAD and LCx.

In a fourth aspect of the present disclosure, which may be combined with any other aspect, or portion thereof, a longest cross-sectional length of the saline/contrast solution lumen is commensurate with at least one of the first and second guidewire diameters.

In a fifth aspect of the present disclosure, which may be combined with any other aspect, or portion thereof, a widest cross-sectional width of the saline/contrast solution lumen is twenty to fifty percent of the longest cross-sectional length of the saline/contrast solution lumen.

In a sixth aspect of the present disclosure, which may be combined with any other aspect, or portion thereof, a left coronary artery (“LCA”) stent system includes: a left main (“LM”) stent assembly including an LM stent sized to be deployed within a patient's LM; a left anterior descending artery (“LAD”) stent assembly including an LAD stent sized differently than the LM stent and to be deployed within the patient's LAD; and a circumflex artery (“LCx”) stent assembly including an LCx stent sized differently than the LM stent and to be deployed within the patient's LCx.

In a seventh aspect of the present disclosure, which may be combined with any other aspect, or portion thereof, the LCx stent is sized differently than the LAD stent.

In an eighth aspect of the present disclosure, which may be combined with any other aspect, or portion thereof, the LM stent is sized to be deployed within the patient's LM only.

In a ninth aspect of the present disclosure, which may be combined with any other aspect, or portion thereof, the LM stent is sized to be deployed within the patient's LM and at least a portion of a transition section in which the LM branches into the LAD and LCx.

In a tenth aspect of the present disclosure, which may be combined with any other aspect, or portion thereof, the LAD stent and the LCx stent each have a different diameter than the LM stent.

In an eleventh aspect of the present disclosure, which may be combined with any other aspect, or portion thereof, the LAD stent and the LCx stent each have a different length than the LM stent.

In a twelfth aspect of the present disclosure, which may be combined with any other aspect, or portion thereof, a left main (“LM”) stent assembly for left coronary artery (“LCA”) cardiovascular treatment includes: a catheter; a balloon attached to the catheter and configured to be inflated; and a stent residing about the balloon and the catheter, the stent sized such that when the balloon is inflated, the stent has a length from 6 mm to 24 mm and a diameter from 4 mm to 6 mm.

In a thirteenth aspect of the present disclosure, which may be combined with any other aspect, or portion thereof, the catheter includes a lumen sized to fit over two guidewires.

In a fourteenth aspect of the present disclosure, which may be combined with any other aspect, or portion thereof, a left coronary artery (“LCA”) stent method includes: deploying a left main (“LM”) stent in a patient's LM such that the LM stent has a first open end and a second open end separated by a plurality of stent struts; inserting at least one of a left anterior descending artery (“LAD”) stent assembly or a circumflex artery (“LCx”) stent assembly through the first opening and within the plurality of stent struts, through the second opening, and into at least one of the patient's LAD or LCx, respectively; and inflating a balloon of the at least one of the LAD or LCx stent assembly to deploy a stent of the at least one LAD or LCx stent assembly.

In a fifteenth aspect of the present disclosure, which may be combined with any other aspect, or portion thereof, the method incudes (i) inserting the LAD stent assembly through the first opening, within the plurality of stent struts, through the second opening, and into the patient's LAD, and inflating the balloon of the LAD assembly to deploy the LCx stent before or after (ii) inserting the LCx stent assembly through the first opening, within the plurality of stent struts, through the second opening, and into the patient's LCx, and inflating the balloon of the LCx assembly to deploy the LCx stent.

In a sixteenth aspect of the present disclosure, which may be combined with any other aspect, or portion thereof, the method further includes removing a stent catheter and the balloon of the stent assembly having the stent deployed in (i) before inserting the stent assembly in (ii).

In a seventeenth aspect of the present disclosure, which may be combined with any other aspect, or portion thereof, the method further includes leaving in the LAD or the LCx a stent catheter and the balloon of the stent assembly having the stent deployed in (i) while inserting the stent assembly in (ii).

In a eighteenth aspect of the present disclosure, which may be combined with any other aspect, or portion thereof, the method includes reinflating the balloon inflated in (i) to correct a deformation of the stent deployed in (i) due to the deployment of the stent in (ii).

In a nineteenth aspect of the present disclosure, which may be combined with any other aspect, or portion thereof, deploying the LM stent includes inserting a catheter carrying the LM stent over a plurality of guidewires.

In a twentieth aspect of the present disclosure, which may be combined with any other aspect, or portion thereof, the LM stent is sized to be deployed (i) in the LM only or (ii) within the patient's LM and at least a portion of a transition section where the LM branches into the LAD and LCx.

In a twenty-first aspect of the present disclosure, which may be combined with any other aspect, or portion thereof, the stent method includes deploying the LM stent and the at least one of the LAD or LCx stent such that a distal end of the LM stent extends over a proximal end of the at least one of the LAD or LCx stent.

In a twenty-second aspect of the present disclosure, which may be combined with any other aspect, or portion thereof, the method includes deploying the LAD and LCx stents at least substantially simultaneously.

In a twenty-third aspect of the present disclosure, which may be combined with any other aspect, or portion thereof, any of the structure and functionality disclosed in connection with FIGS. 18 to 30 may be combined with any of the other structure and functionality disclosed in connection with FIGS. 18 to 30.

In light of the present disclosure and the above aspects, it is therefore an advantage of the present disclosure to provide a left coronary artery (“LCA”) stent system and associated methodology, which does not require the crushing of a previously deployed stent.

It is another advantage of the present disclosure to provide an LCA stent system and associated methodology, which does not require the threading of guidewires through one or more strut of a previously deployed stent.

It is a further advantage of the present disclosure to provide an LCA stent system and associated methodology, which does not require the threading of ballon stent catheters through one or more strut of a previously deployed stent.

It is yet another advantage of the present disclosure to provide an LCA stent system and associated methodology, which is less susceptible to stent renarrowing.

It is yet a further advantage of the present disclosure to provide an LCA stent system and associated methodology, which does not require (but may employ) rewiring.

Still another advantage of the LCA stent system and associated methodology of the present disclosure is to provide a large stented access in the left main (“LM”) for the deployment of branch stents into one or both of the left anterior descending artery (“LAD”) or the circumflex artery (“LCx”).

Still a further advantage of the present disclosure is to provide an LCA stent system and associated methodology that allows each deployed stent to be maintained in its intended cylindrical shape.

Yet another advantage of the present disclosure is to provide an LCA stent system and associated methodology that allows stents to be sized optimally for each of the LM, LAD and LCx.

The advantages discussed herein may be found in one, or some, and perhaps not all of the embodiments disclosed herein. Additional features and advantages are described herein, and will be apparent from, the following Detailed Description and the figures.

### BRIEF DESCRIPTION OF THE FIGURES

FIG. 1 is a front elevation prior art view of a human heart illustrating that in most instances, the left main (“LM”) of the left coronary artery (“LCA”) bifurcates into two branches, namely, the left anterior descending artery (“LAD”) and the circumflex artery (“LCx”).

FIG. 2 is a front elevation view of a prior art Dkcrush technique step, illustrating the LM branching into the LAD and the LCx, each of which are diseased with plaque build-up.

FIG. 3 is a front elevation view of a prior art Dkcrush technique step, illustrating guidewires inserted into the LAD and LCx.

FIG. 4 is a front elevation view of a prior art Dkcrush technique step, illustrating a first stent advanced along an LCx guidewire into the LCx.

FIG. 5 is a front elevation view of a prior art Dkcrush technique step, illustrating a balloon advanced along the LM and LAD guidewire into the LM and LAD.

FIG. 6 is a front elevation view of a prior art Dkcrush technique step, illustrating a balloon within the LCx stent inflated to deploy the LCx stent.

FIG. 7 is a front elevation view of a prior art Dkcrush technique step, illustrating the catheter and balloon removed from the deployed LCx stent along the LCx guidewire, leaving only the deployed LCx stent, the LCx guidewire and the LM/LAD balloon and its associated guidewire.

FIG. 8 is a front elevation view of a prior art Dkcrush technique step, illustrating the balloon introduced in FIG. 5 into the LM and LAD inflated to crush the upper, proximal corner of the deployed LCx stent.

FIG. 9 is a front elevation view of a prior art Dkcrush technique step, illustrating two new balloons inserted, namely, a first balloon inserted though the crushed struts of the LCx stent, and a second balloon inserted into the LM and LAD.

FIG. 10 is a front elevation view of a prior art Dkcrush technique step, illustrating the two new balloons of FIG. 9 inflated, such that the two balloons touch and perform a “first kiss”.

FIG. 11 is a front elevation view of a prior art Dkcrush technique step, illustrating a second stent advanced along the LM and LAD guidewires into the LM and LAD.

FIG. 12 is a front elevation view of a prior art Dkcrush technique step, illustrating the LM and LAD stents inflated for deployment in the LM and LAD.

FIG. 13 is a front elevation view of a prior art Dkcrush technique step, illustrating the catheters and balloons removed from the deployed LM and LAD stents, leaving only the deployed LCx and LM/LAD stents and their respective guidewires.

FIG. 14 is a front elevation view of a prior art Dkcrush technique step, illustrating two additional new balloons inserted, namely, a first additional balloon inserted through the struts of the LM and LAD stent and the crushed struts of the LCx stent, while a second additional balloon is inserted into the LM and LAD.

FIG. 15 is a front elevation view of a prior art Dkcrush technique step, illustrating the two additional new balloons inflated, such that the two additional balloons touch, performing the “second kiss”.

FIG. 16 is a front elevation view of the outcome of a prior art Culotte LM stent technique.

FIG. 17 is a front elevation view of the outcome of a prior art LM stent technique used on hemodynamically unstable patients.

FIG. 18 is a side elevation view of one embodiment of a left coronary artery (“LCA”) stent system of the present disclosure.

FIG. 19A is a sectioned front view taken along line XIXA-XIXA of FIG. 18 illustrating an example of a guidewire lumen large enough to accept two guidewires and a saline/contrast solution lumen for balloon inflation existing along a same LM stent catheter of the present discsloure.

FIG. 19B is a perspective view of a section of the LM catheter at which the saline/contrast solution lumen fluidly communicates with and expands the balloon of the balloon catheter.

FIG. 20 is a front elevation view of one step of an embodiment of the methodology associated with the LCA stent system of the present disclosure, in which guidewires are inserted through the LM and into the LAD and LCx.

FIG. 21 is a front elevation view of another step of an embodiment of the methodology associated with the LCA stent system of the present disclosure, in which, for example, disease in a distal portion of the LM and a proximal portion of the LAD is pretreated.

FIG. 22 is a front elevation view of a further step of an embodiment of the methodology associated with the LCA stent system of the present disclosure, in which, for example, disease in the LCx is pretreated.

FIG. 23 is a front elevation view of yet another step of an embodiment of the methodology associated with the LCA stent system of the present disclosure, in which an LM stent is introduced into the patient's LM over the two inserted guidewires.

FIG. 24 is a front elevation view of yet a further step of an embodiment of the methodology associated with the LCA stent system of the present disclosure, in which the LM stent is inflated and deployed within the patient's LM.

FIG. 25 is a front elevation view of still another step of an embodiment of the methodology associated with the LCA stent system of the present disclosure, in which an LAD stent is introduced through the deployed LM stent and into the patient's LM over the inserted LAD guidewire.

FIG. 26 is a front elevation view of still a further step of an embodiment of the methodology associated with the LCA stent system of the present disclosure, in which the LAD stent is inflated and deployed within the patient's LAD.

FIG. 27 is a front elevation view of yet still another step of an embodiment of the methodology associated with the LCA stent system of the present disclosure, in which an LCx stent is introduced through the deployed LM stent and into the patient's LCx over the inserted LCx guidewire.

FIG. 28 is a front elevation view of yet still a further step of an embodiment of the methodology associated with the LCA stent system of the present disclosure, in which the LCx stent is inflated and deployed within the patient's LCx.

FIG. 29 is a front elevation view of one completed LCA multi-stent deployment performed using the system and methodology of the present disclosure.

FIG. 30 is a front elevation view of another completed LCA multi-stent deployment performed using the system and methodology of the present disclosure.

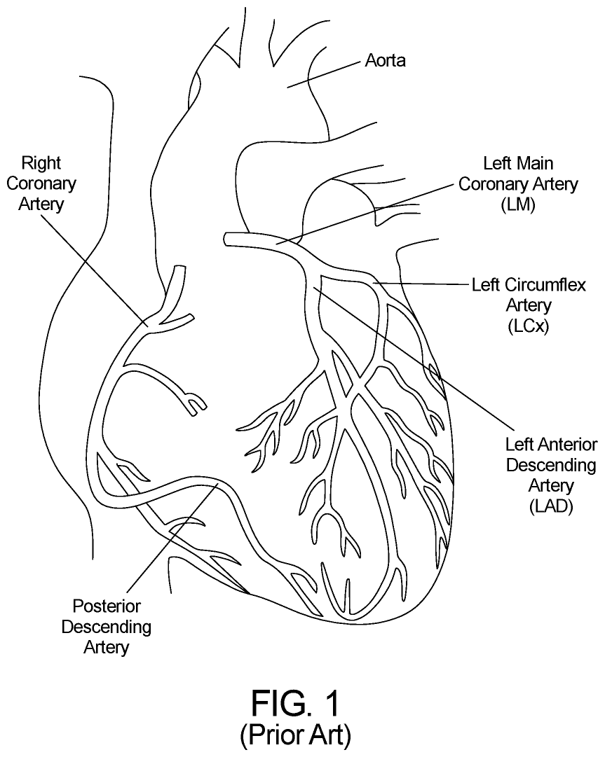

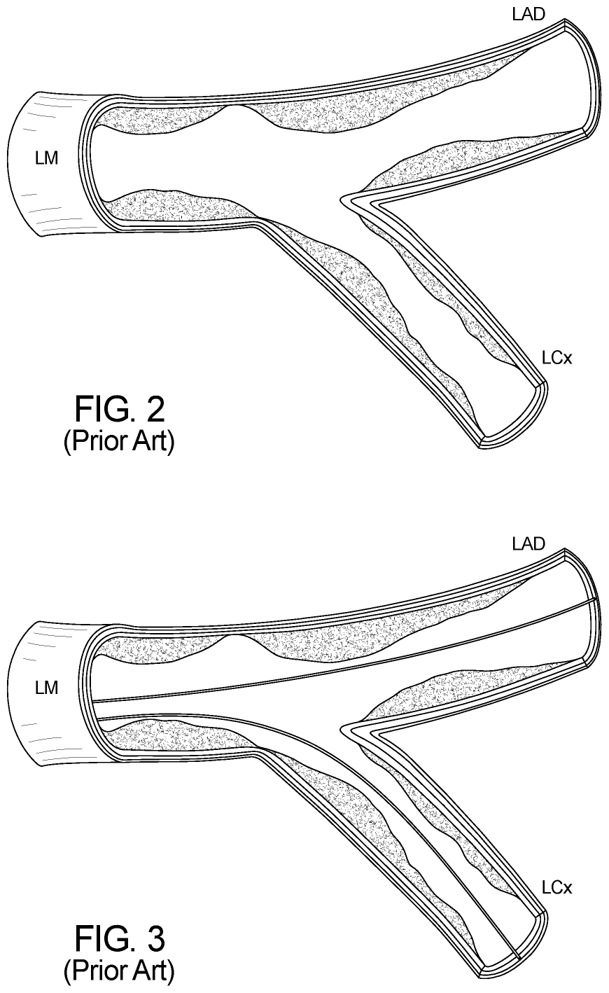

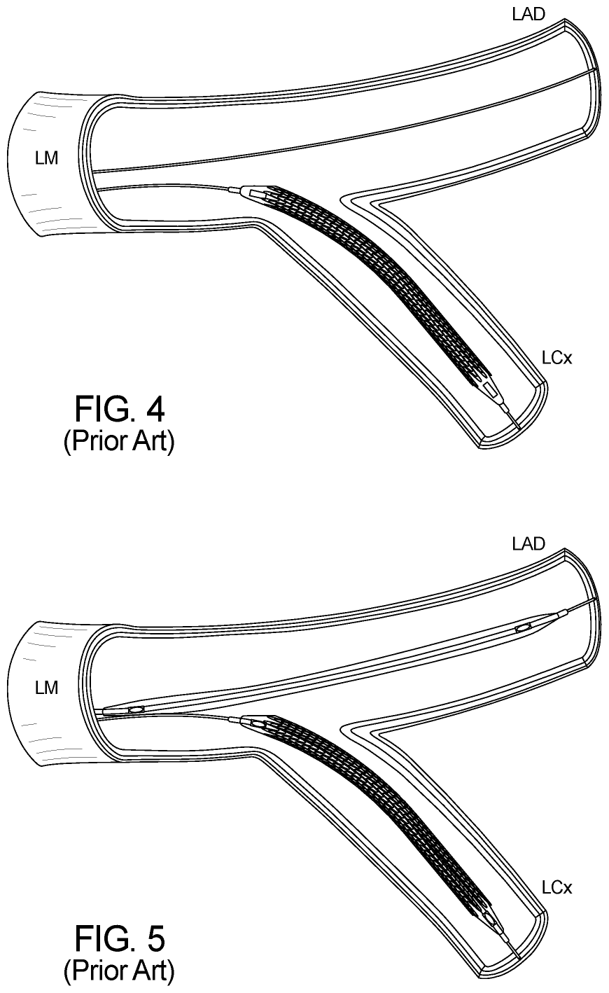

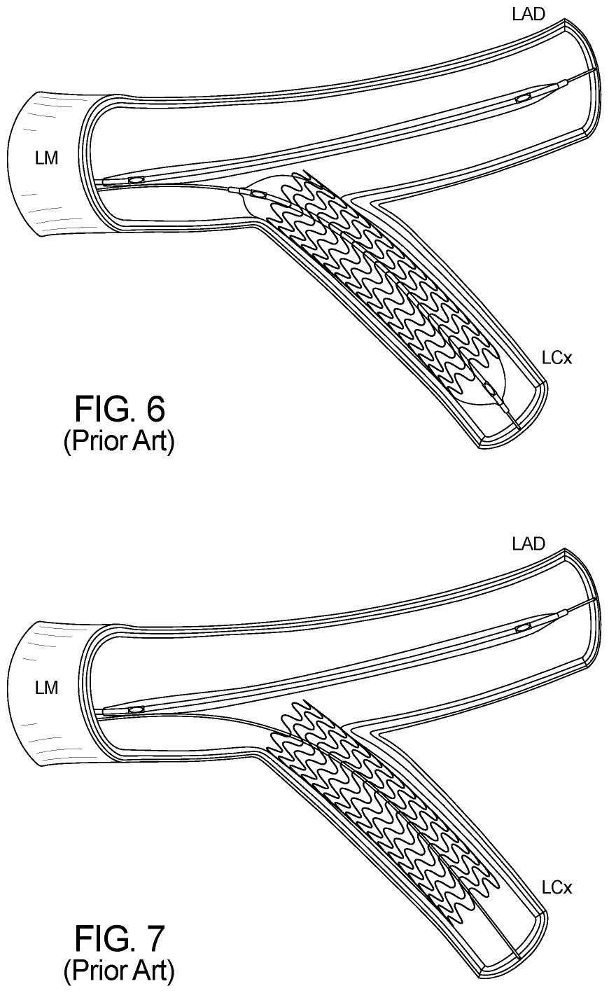

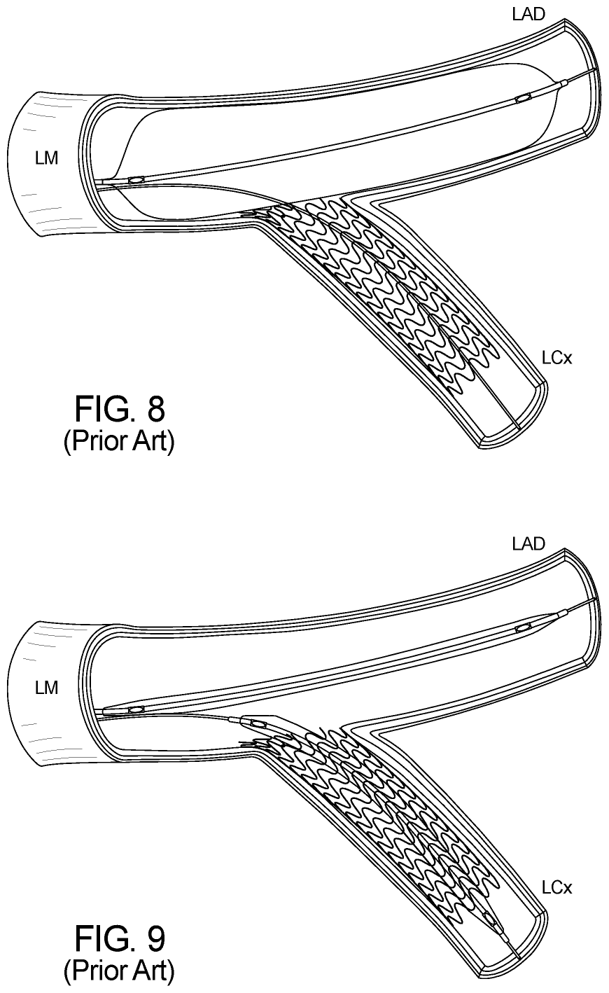

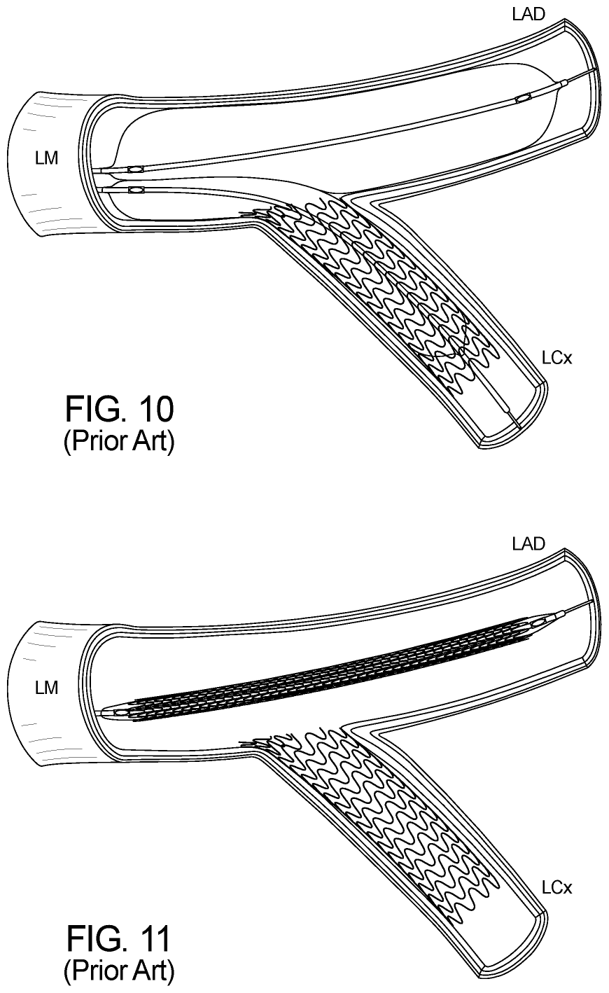

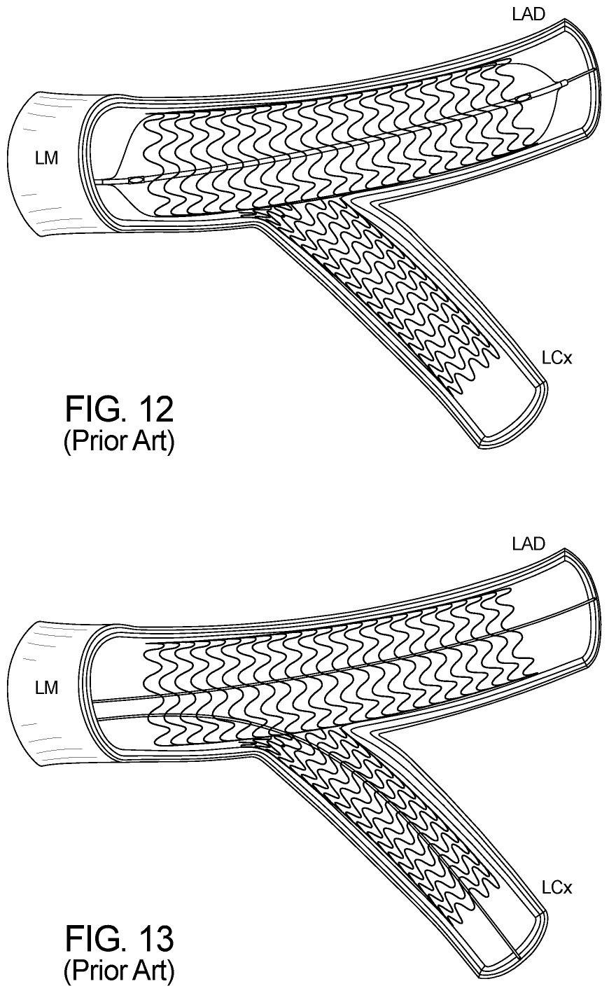

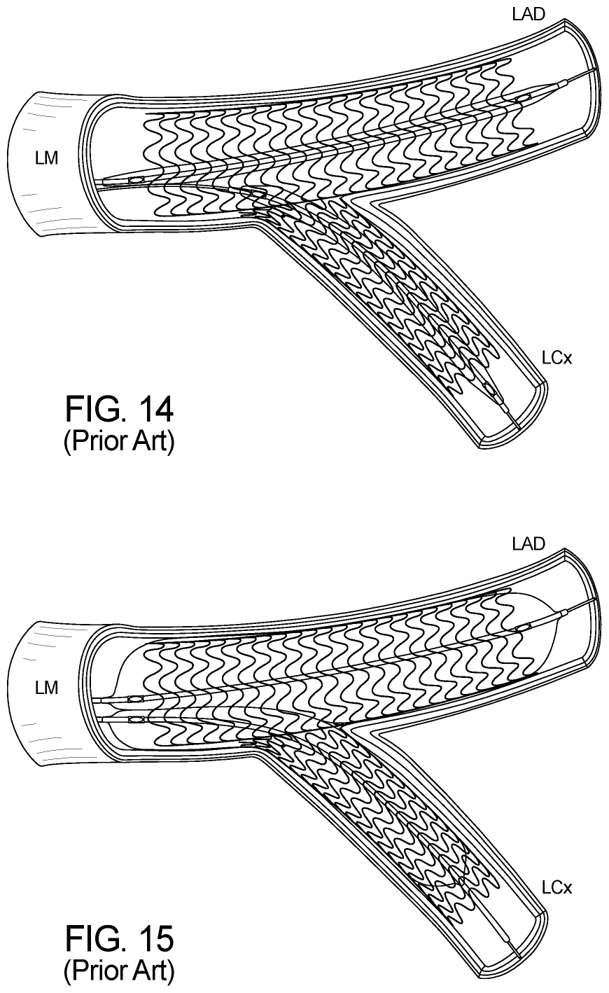

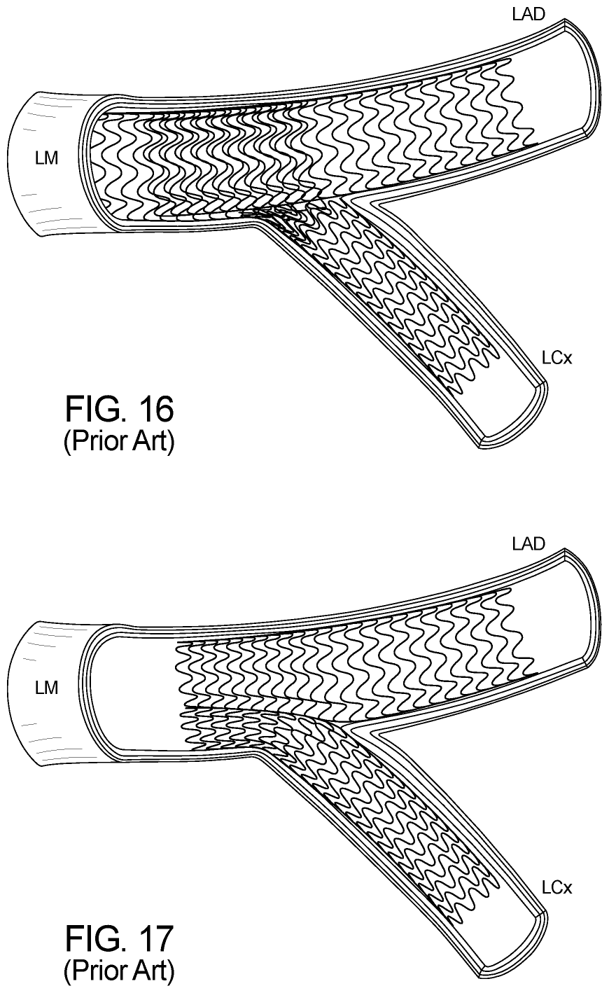

### DETAILED DESCRIPTION

### System Overview

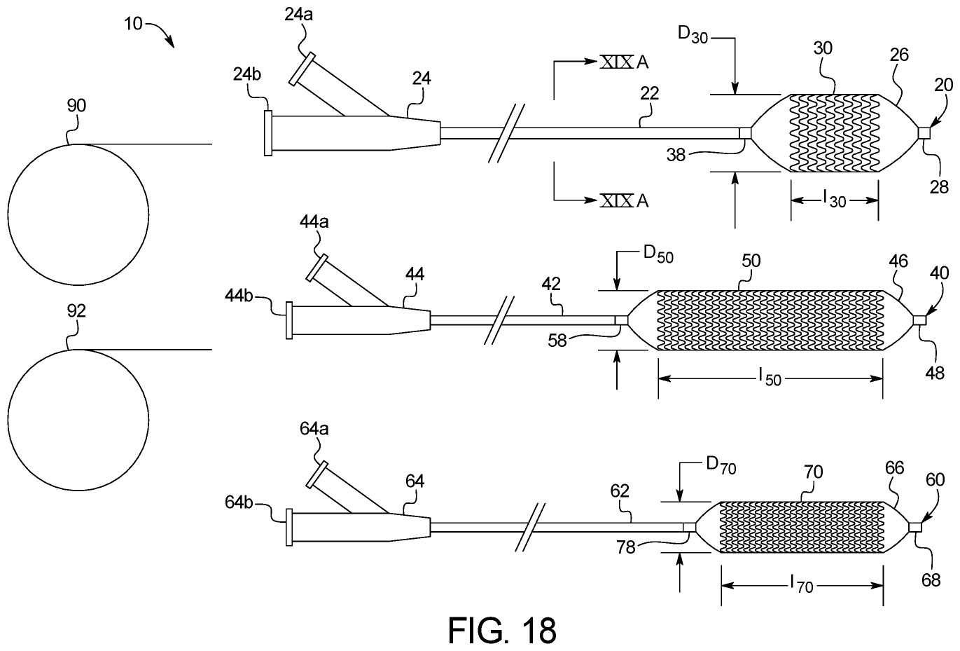

Referring now to the drawings and in particular to FIG. 18, one embodiment of a left coronary artery (“LCA”) stent system 10 is illustrated. LCA stent system 10 in the illustrated embodiment is provided with three balloon catheter stent assemblies, namely, a left main (“LM”) balloon catheter stent assembly 20, a left anterior descending artery (“LAD”) balloon catheter stent assembly 40, and a circumflex artery (“LCx”) stent assembly 60. LCA stent system 10 is provided with at least one, and in the illustrated embodiment two guidewires 90 and 92.

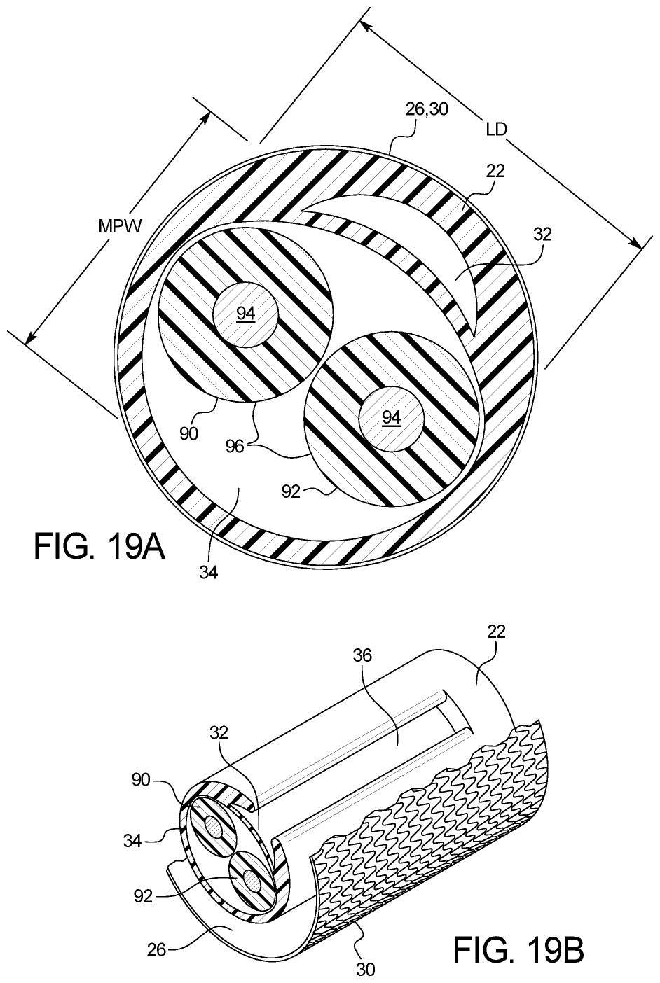

In the illustrated embodiment, LM stent assembly 20 includes a catheter 22. Catheter 22 extends from a distal end of a connector 24, e.g., a Y-connector in the illustrated embodiment, to a distal end 28 of LM stent assembly 20. Catheter 22 may be made of any known material for the catheter, such as silicone or polyurethane, while distal end 28 may be provided with or as a radiopaque tip, which is opaque to one or more or all forms of radiation, such as X-rays or other electromagnetic radiation. Radiopaque tip 28 blocks radiation rather than allowing it to pass through the tip. In this manner, the doctor can see the location of distal end 28 of catheter 22 within the patient during operation via an x-ray machine. A second radiopaque marker 38 is located in the illustrated embodiment at a proximal end of balloon 26, so that the balloon and stent 30 may be centered at a desired location for actuation. Catheter 22 may have any desired length, such as 120 cm to 160 cm. Suitable diameter dimensions for catheter 22 of LM stent assembly 20 are discussed in connection with FIG. 19A.

As illustrated, while connector 24 is provided on a proximal end of LM stent assembly 20, a balloon 26 is provided on the distal end of LM stent assembly 20, adjacent to radiopaque tip 28. Balloon 26 in various embodiments is made of nylon, Pebax™, polyethylene terephthalate (“PET”), polyurethanes, polymer blends, and may comprise a single or multiple layers. Balloon 26 has a length commensurate with the stent 30 that the balloon inflates. In the illustrated embodiment, balloon 26 is somewhat longer (exaggerated for illustration) than stent 30 and its ends may be visible when the balloon is inflated.

Connector 24 may be made of any material listed herein or of polyvinyl chloride (“PVC”), acrylonitrile butadiene styrene (“ABS”) or other suitable polymer. Connector 24 in the illustrated embodiment is a Y-connector having two ports 24a and 24b, which may for example be female luer connectors. Port 24a communicates with a pressurized saline/contrast fluid delivery device (not illustrated), which is a reusable item and is accordingly not packaged with the other components of system 10 in one embodiment. The pressurized saline/contrast fluid delivery device is capable of generating the specified pressure needed to inflate balloon 26 with saline/contrast fluid and deploy stent 30, which may be in the range of 507 kPa to 2027 kPa (five to twenty atmospheres). Port 24a leads to and is in sealed fluid communication with a thin saline/contrast solution lumen 32 (see FIG. 19A), which is placed into sealed saline/contrast solution communication with the pressurized saline/contrast solution delivery device. Saline/contrast solution lumen 32 extends within catheter 22 to balloon 26, so that pressurized saline/contrast solution may be delivered from the pressurized saline/contrast solution delivery device, through saline/contrast solution lumen 32, to balloon 26 when it is desired to inflate balloon 26 and deploy stent 30. The contrast fluid also aids in detecting radioopaque markers 38.

Port 24b of LM stent assembly 20 is sized to accept two guidewires 90 or 92, which may each be 14/1000 inch (0.36 mm) in outer diameter, but may alternatively be anywhere from and including 10/1000 inch (0.25 mm) in outer diameter to 25/1000 inch (0.64 mm) in outer diameter. The diameters of guidewires 90 and 92 may be the same or different. Guidewires 90 or 92 as shown in detail below are able to extend into the patient's blood vessels so as to provide guidance later for stent assembly 20 and stent assemblies 40 and 60 discussed below, wherein the catheters 22, 42 and 62 respectively of assemblies 20, 40 and 60 are inserted over a proximal end of one or both of the guidewires and slid along the guidewires to a desired location for stent deployment.

Stent 30 in the illustrated embodiment includes struts, lattices or meshes of metal or metal alloy. Stent 30 (and any of the stents herein) may be bare metal or be drug-eluting, in which the stent is coated with a time-release, anti-clotting drug to protect the deployment site. Suitable metals or alloys for stent 30 include cobalt, nitinol (shape memory), platinum, stainless steel, one of a plurality of titanium metals or alloys thereof

In FIG. 18, stent 30 has a length l₃₀ and a deployed diameter D₃₀ (which may be an inside diameter). It is contemplated that LM stent 30 be relatively wide in diameter and short in length so as to fit into the patient's LM without extending significantly into the LAD, LCx or the patient's aorta. Suitable diameters for D₃₀ include 4 mm to 6 mm, while suitable lengths for l₃₀ include 6 mm to 24 mm. The larger diameter D₃₀ is extended into the wider vessel wall of the patient's LM and provides a large access for the branch stent assemblies as discussed below.

In the illustrated embodiment of FIG. 18, LAD balloon catheter stent assembly 40 includes connector 44, which includes all structure, functionality and alternatives described for connector 24, except connector 44 of stent assembly 40 only needs to be sized to slide over one guidewire 90 or 92 as illustrated below. Pressurized saline/contrast solution delivery port 44a of connector 44 delivers saline/contrast solution to inflate balloon 46, while port 44b of connector 44 is sized to accept one of guidewires 90 or 92. LAD stent assembly 40 may also include a catheter 42 having radioopaque distal end 48, balloon 46, stent 50, and proximal radioopaque marker 58, which have all the structure, functionality and alternatives described respectively for catheter 22 having radioopaque distal end 28, balloon 26, stent 30, and proximal radioopaque marker 38 for LM stent assembly 20.

Catheter 42 and connector 44 of LAD stent assembly 40 as illustrated are smaller in diameter than catheter 22 and connector 24 of LM stent assembly 20. As illustrated below, catheter 42 only needs to fit over one guidewire 90 or 92, so that the catheter diameter may be smaller. In an embodiment, the diameter (which may be the outer diameter) of catheter 42 is two French (“Fr”) or 0.67 mm.

Balloon 46 and stent 50 of LAD stent assembly 40 are shaped differently than balloon 26 and stent 30 of LM stent assembly 20. The diameter and length of balloon 46 are sized according to length l₅₀ and a deployed diameter D₅₀ (which may be an inside diameter) of stent 50. It is contemplated that LAD stent 50 be relatively small in diameter and longer in length than LM stent 30, so as to fit as needed into the smaller diameter LAD of the patient and to fully cover the disease within same. Suitable diameters for D₅₀ include 2.0 mm to 3.5 mm, while suitable lengths for l₅₀ include 10 mm to 38 mm.

In the illustrated embodiment of FIG. 18, LCx balloon catheter stent assembly 60 includes connector 64, which includes all structure, functionality and alternatives described for connector 24, except connector 64 of stent assembly 60 only needs to be sized to accept one guidewire 90 or 92 as illustrated below. Pressurized saline/contrast solution delivery port 64a of connector 64 delivers saline/contrast solution to inflate balloon 66, while port 64b of connector 64 is sized to accept one of guidewires 90 or 92. LCx stent assembly 60 may also include a catheter 62 having radioopaque distal end 68, balloon 66, stent 70, and proximal radioopaque marker 78 having all the structure, functionality and alternatives described respectively for catheter 22 having radioopaque distal end 28, balloon 26, stent 30, and radioopaque marker 38 for LM stent assembly 20.

Catheter 62 and connector 64 of LCx stent assembly 60 as illustrated are smaller in diameter than catheter 22 and connector 24 for LM stent assembly 20. As illustrated below, catheter 62 only needs to fit over one of guidewires 90 or 92, so that the catheter diameter may be smaller. In an embodiment, the diameter (which may be the outer diameter) of catheter 62 is two Fr or 0.67 mm, like catheter 42 of LAD stent assembly 40.

Balloon 66 and stent 70 of LCx stent assembly 60 are shaped differently than balloon 26 and stent 30 of LM stent assembly 20 (and possibly balloon 46 and stent 50 of LM stent assembly 40). The diameter and length of balloon 66 are sized according to length l₇₀ and a deployed diameter D₇₀ (which may be an inside diameter) of stent 70.

It is contemplated that LCx stent 70 be relatively small in diameter and longer in length than LM stent 30, so as to fit as needed into the smaller diameter LCx of the patient and to fully cover the disease within same. Suitable diameters for D₇₀ include 1.5 mm to 3.5 mm, while suitable lengths for 170 include 10 mm to 38 mm.

FIG. 18 further illustrates that LCA stent system 10 includes guidewires 90 and 92. Guidewires 90 and 92 in one embodiment may each be 14/1000 inch (0.36 mm) in outer diameter, but may alternatively be sized anywhere from and including 10/1000 inch (0.25 mm) in outer diameter to 25/1000 inch (0.64 mm) in outer diameter.

System 10 may be packaged in different ways. In one embodiment, all three stent assemblies 20, 40 and 60, along with their plastic tubing covers (not illustrated) and guidewires 90 and 92, are provided in a single package. In another embodiment, stent assemblies 20, 40 and 60, along with their plastic tubing covering (not illustrated) are each provided in their own individual package, each having at least one guidewire 90 or 92. In a further alternative embodiment, stent assemblies 20 and 40 or 20 and 60, along with their plastic tubing covers (not illustrated) and guidewires 90 and 92, are provided in a single package, while a third (or fourth, e.g., for a third or fourth branch off of the LM) stent assembly 40 or 60, along with its plastic tubing covering (not illustrated) and guidewire 90 or 92, is provided as needed in its own individual package.

Referring now to FIG. 19A, a guidewire lumen 34 of catheter 22 of LM stent assembly 20 of FIG. 18 is illustrated having been slid over guidewires 90 and 92. Catheter 22 also includes a thin saline/contrast solution lumen 32 that coexists and coextends within the same catheter 22 of LM stent assembly 20. In one embodiment, guidewires 90 and 92 are 14/1000 inch in diameter, which is 0.36 mm. Two of such wires placed side-by-side therefore span a collective distance of about 0.72 mm. Guidewire lumen 34 therefore has a longest dimension LD of at least 0.72 mm and a maximum perpendicular width MPW of at least 0.36 mm (MPW is illustrated in FIG. 19A as being significantly larger than 0.36 mm), each of which fit within a 4 Fr catheter that typically has an outer diameter of 1.35 mm. As illustrated in FIG. 19A, the 4 Fr catheter provides plenty of cross-sectional wall space within which to co-extrude saline/contrast solution lumen 32 along with the above-dimensioned guidewire lumen 34. Thinner guidewires may be used to allow for thinner LM catheters, and different catheter materials may require different wall thicknesses, such that LM catheter 22 may be of a 3 Fr size or larger than a 4 Fr size. As shown below, the relatively large diameter of deployed LM stent 30 is advantageous to overall system 10 because catheter 22 is correspondingly relatively large, facilitating the extension of guidewires 90 and 92 through an interior of the catheter.

Saline/contrast solution lumen 32 as discussed above is placed in sealed fluid communication at its proximal end with pressurized saline/contrast solution delivery port 24a of connector 24. Pressurized saline/contrast solution delivery port 24a is connected to or placed in saline/contrast solution communication with a pressurized saline/contrast solution delivery source for pressurizing saline/contrast solution lumen 32 to open balloon 26. The cross-section XIXA-XIXA of catheter 22 taken in FIG. 18 is at a location of the catheter that has not yet reached balloon 26 or stent 30. Balloon 26 and stent 30 are still visible in FIG. 19A due to the direction of cross-section arrows XIXA, which point to the right. But balloon 26 and stent 30 are not sectioned in FIG. 19A because they reside further down the distal end of catheter 22.

The example saline/contrast solution lumen 32 in FIG. 19A is illustrated as having a crescent or half-moon shape to efficiently use space within the wall of catheter 22 alongside the somewhat elliptical or oval shape of guidewire lumen 34. Saline/contrast solution lumen 32 may have different shapes, such as an arc shape, e.g., with rounded ends or a straight shape, e.g., with rounded ends. In the illustrated embodiment, saline/contrast solution lumen 32 may have a longest cross-sectional length (corner to corner) commensurate with the diameters of guidewires 90 and 92 and a widest cross-sectional width (curve to curve) that is twenty to fifty percent of the longest length of lumen 32. Saline/contrast solution lumen 32 may be relatively thin because the amount of saline/contrast solution needed to expand balloon 26 is very small.

Regardless of the shape of saline/contrast solution lumen 32, FIG. 19B illustrates that when lumen 32 extends to and reaches the portion of catheter 22 that is in registry with balloon 26 and stent 30, lumen 32 is fluidly communicated with the inside of balloon 26, e.g., via a channel 36 extending from the crescent, arc or straight shape lumen 32 through the reminder of the wall of catheter 22. Channel 36 allows saline/contrast solution to flow from the crescent, arc or straight shape lumen 32 into the interior of balloon 26 to inflate same. Channel 36 may alternatively be a series of holes other type or shape of fluid passageway. Catheters 42 and 62 of LAD stent assembly 40 and LCx stent assembly 60, respectively, have a like series of holes extending to saline/contrast solution lumens to inflate their respective balloons 46 and 66.

Thicker or thinner guidewires 90 or 92 may be used to allow for thicker or thinner LM catheters 22 and different catheter materials may require different wall thicknesses. The above numbers are therefore meant to provide one working example. For example, if needed to accommodate the outer diameters of larger guidewires 90 and 92, a 5 Fr (1.67 mm outside diameter) to an 8 Fr (2.7 mm outside diameter) LM catheter 22 may be provided.

As illustrated in FIG. 19A, guidewires 90 and 92 may each include a metal core 94 surrounded by a polymer coating 96. The metal core may be gold, nitinol (“NiTi”) a nickel-titanium alloy (which has exceptional shape-memory, but limited rigidity), platinum, stainless steel, stainless steel with nickel, titanium, tungsten or alloys thereof Coating 96 may be polytetrafluoroethylene (“PTFE”), silicone, or tetrafluoroethylene (“TFE”). The length of guidewires 90 and 92 is as needed depending on where the patient access resides.

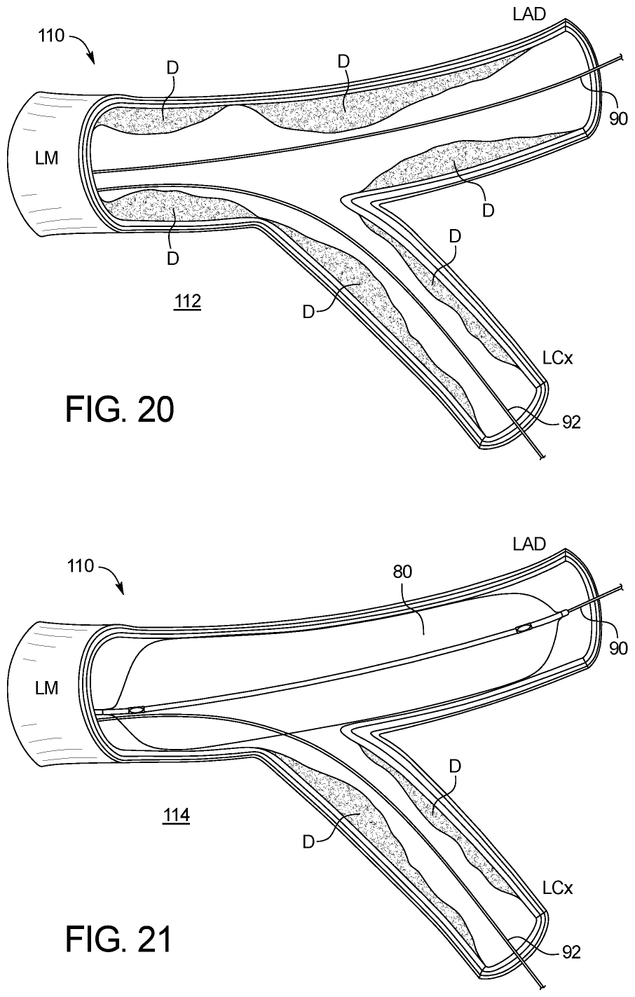

Referring now to FIGS. 20 to 30, method 110 illustrates various embodiments for treating left coronary artery (“LCA”) disease using stent system 10 described herein. In FIG. 20 at pretreatment step 112, guidewires 90 and 92 are inserted through the patient's LM and into the LAD and LCx, respectively. Guidewires 90 and 92 may be 14/1000 inch in diameter, or 0.36 mm, and be constructed according to the embodiments described above. Guidewires 90 and 92 are inserted respectively as far as desired into the patient's LAD and LCx. FIG. 20 illustrates that the patient has disease D in all three of the LM, LAD and LCx.

In FIG. 21 at pretreatment step 114, a separate balloon 80 (not a stent balloon) is inserted along guidewire 90 into a distal portion of the patient's LM and the diseased portion of the patient's LAD. The pressurized saline/contrast solution delivery device connected to port 24a of connector 24 (FIG. 18) is used to pressurize balloon 80 to a specified pressure, compressing disease D in the LM and LAD as illustrated in FIG. 21. Afterwards, balloon 80 is removed along guidewire 90.

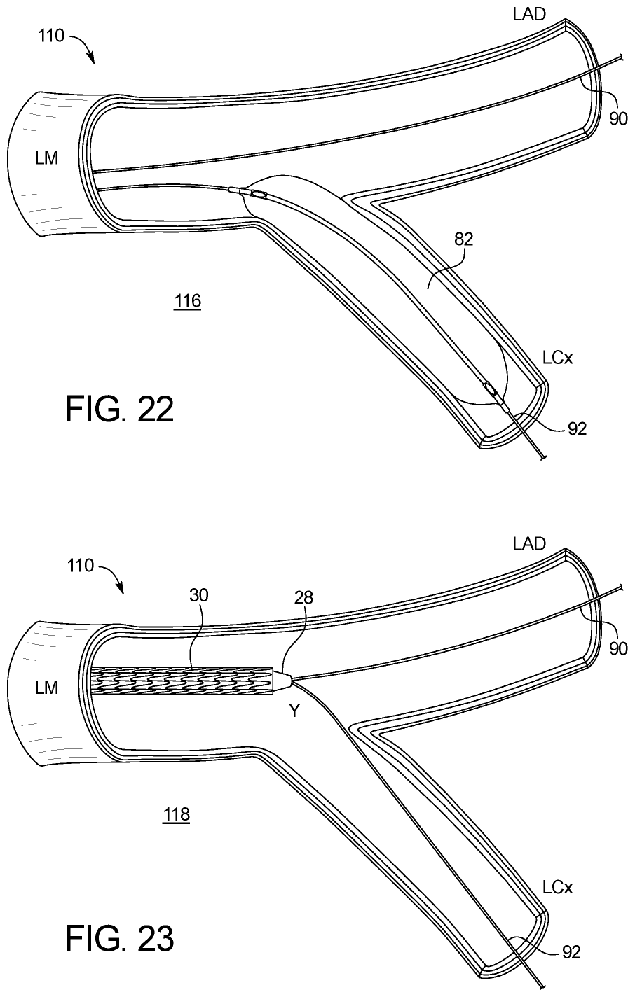

In FIG. 22 at pretreatment step 116, a second separate balloon 82 is inserted along guidewire 92 into the diseased portion of the patient's LCx. The pressurized saline/contrast solution delivery device connected to port 24a of connector 24 (FIG. 18) is used to pressurize balloon 82 to a specified pressure, compressing disease D in the LCx as illustrated in FIG. 22. Afterwards, balloon 82 is removed along guidewire 92. It should be appreciated that either one or both steps 114 and 116 is optional, and may not be performed depending on the extent of disease D and/or preference of the physician. Steps 114 and 116 may also be reversed.

In FIG. 23 at step 118, LM stent 30 of LM stent assembly 20 is inserted along both guidewires 90 and 92 so as to reside at a desired location within the patient's LM. In the illustrated embodiment, radiopaque tip 28 allows the doctor to position the distal end of LM stent 30 to a threshold of, or slightly into, a transition section Y, where the LM branches or splits into the LAD and the LCx. Doing so as illustrated below allows the proximal ends of the LAD stent 50 and LCx stent 70 to ultimately reside within the distal end of LM stent 30. Guidewire lumen 34 of catheter 22 is sized appropriately with respect to the diameters of guidewires 90 and 92 and taking into account its saline/contrast solution lumen 32, so that catheter 22 may slide readily over the guidewires.

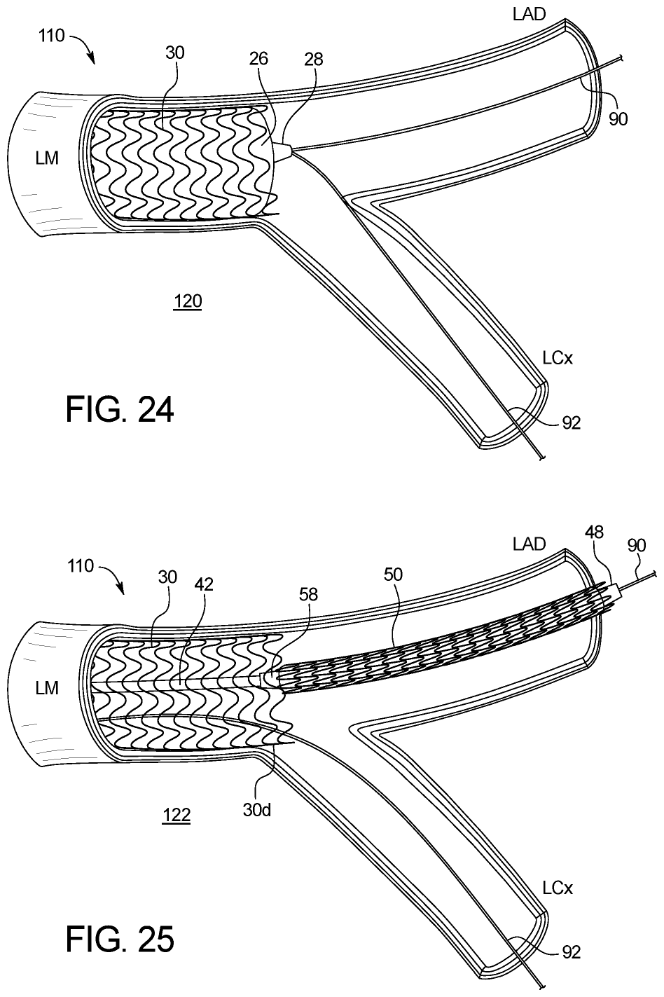

In FIG. 24 at step 120, the pressurized saline/contrast solution delivery device connected to port 24a of connector 24 is used to pressurize balloon 26 of LM stent assembly 20 to a specified pressure, opening and deploying LM stent 30. LM stent 30 is sized and positioned as described above to extend the threshold of, or slightly into, transition section Y where the LM branches or splits into the LAD and LCx, while also fully dilating the diseased area of the LM vessel wall (but not extending into the patient's aorta). At step 120, guidewires 90 and 92 remain inside the LM, LAD and LCx as illustrated.

In FIG. 25 at step 122, LAD stent 50 of LAD stent assembly 40 is inserted through both open ends and within the struts of deployed LM stent 30, and along guidewire 90 only, so as to reside at a desired location within the LAD. In the illustrated embodiment, proximal radiopaque tip 58 allows the proximal end of LAD stent 50 to be positioned slightly overlappingly within a distal end 30d of LM stent 30. Proximal radiopaque tip 58 may have to be pushed all the way through LM stent 30 temporarily so as to be viewable, before being pulled back a slight distance to provide the desired overlap. LAD stent 50 is sized as needed to properly treat the diseased portion of the LAD.

In FIG. 25, catheter 42 of LAD stent assembly 40 easily fits over single guidewire 90, while the non-inflated LAD stent 50 fits easily within and through deployed LM stent 30. Again, the length and diameter of deployed LAD stent 50 may be selected specifically in accordance with the geometry of the patient's LAD and/or the location of the disease within the LAD.

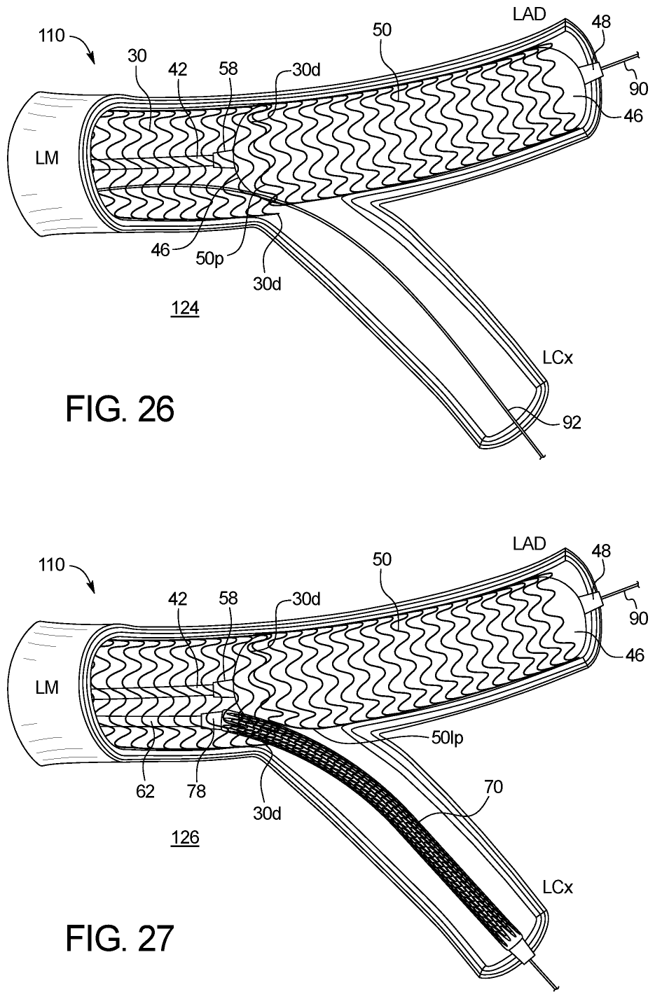

In FIG. 26 at step 124, the pressurized saline/contrast solution delivery device connected to port 44a of connector 44 is used to pressurize balloon 46 of LM stent assembly 40 to a specified pressure, opening and deploying LAD stent 50. The proximal end 50p of LAD stent 50 in the illustrated embodiment is positioned when deployed to reside within the distal end 30d of LM stent 30, so that there is no uncovered blood vessel wall in the transition section Y between the LM and LAD. In the illustrated embodiment both ends of balloon 46 are illustrated along with both radiopaque markers 48 and 58 to highlight the expansion of LM stent 50. As discussed with FIG. 25, LAD stent 50 when deployed extends for a desired length into the patient's LAD to fully treat its disease.

In FIG. 27 at step 126, LCx stent 70 of LCx stent assembly 60 is inserted through both open ends and within the struts of deployed LM stent 30, and along guidewire 92 only, so as to reside at a desired location within the LCx. In the illustrated embodiment, proximal radiopaque tip 78 allows the proximal end of LCx stent 70 to be positioned slightly overlappingly within a distal end 30d of LM stent 30. Proximal radiopaque tip 78 may have to be pushed all the way through LM stent 30 temporarily so as to be viewable, before being pulled back a slight distance to provide the desired overlap. LCx stent 70 is sized as needed to properly treat the diseased potion of the LCx.

In FIG. 27, catheter 62 of LCx stent assembly 60 easily fits over single guidewire 92, and non-inflated stent 70 fits easily within and through deployed LM stent 30. Non-inflated stent 70 as illustrated in FIG. 27 deforms the lower portion of the proximal end 50lp of LAD stent 50, if needed, to move into the LCx. The length and diameter of deployed LCx stent 70 may be selected specifically in accordance with the geometry of the patient's LCx and/or the location of the disease within the LCx.

In the illustrated embodiment, catheter 42 and balloon 46 of LAD stent assembly 40, along with guidewire 90, are left in place so that balloon 46 can be inflated later if needed as a finishing procedure in case either LM stent 30 or LAD stent 50 are deformed during the deployment of LCx stent 70. In an alternative embodiment, catheter 42, balloon 46 and guidewire 90 are removed prior to step 126. Importantly, deployed LM stent 30 provides enough room for the balloons of the branch stents to be left in place in case any corrective action needs to be undertaken once all three stents 30, 50 and 70 have been deployed.

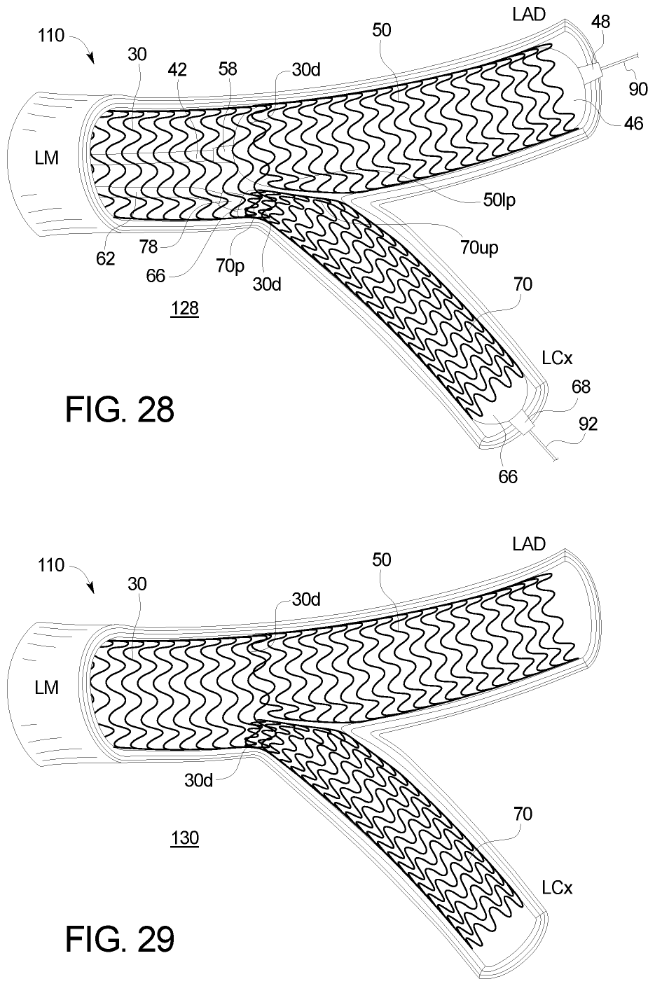

In FIG. 28 at step 128, the pressurized saline/contrast solution delivery device connected to port 64a of connector 64 is used to pressurize balloon 66 of LCx stent assembly 60 to a specified pressure, opening and deploying LCx stent 70. The proximal end 70p of LCx stent 70 in the illustrated embodiment is positioned when deployed to reside within the distal end 30d of LM stent 30, so that there is no uncovered blood vessel wall in the transition section Y between the LM and LCx. In the illustrated embodiment both ends of balloon 66 are illustrated along with both radiopaque markers 68 and 78 to highlight the expansion of LCx stent 70. As discussed with FIG. 27, LCx stent 70 when deployed extends for a desired length into the patient's LCx to fully treat its disease.

As illustrated in FIG. 28, upper portion of proximal end 70up of inflated LCx stent 70 further deforms lower portion of the proximal end 50lp of inflated stent LAD stent 50. If needed and available, LAD balloon 46 and LCx balloon 66 may be inflated at the same time so that the interface between upper portion of proximal end 70up and lower portion of the proximal end 50lp reside roughly halfway within the transition section Y, between the LAD and LCx. The inflation of LAD balloon 46 and LCx balloon 66 either simultaneously or at different times also causes distal end 30d of LM stent 30 to deform outwardly so as to follow the increasing diameter of transition section Y as the LM transitions into the LAD and LCx.

FIG. 29 at step 130 illustrates all three stents 30, 50 and 70 deployed with all catheters 42, 62, balloons 46, 66 and guidewires 90, 92 removed. In FIG. 29, transition section Y and abutting portions of LM, LAD and LCx are fully covered and thereby thoroughly treated. The proximal ends (hidden) of LAD stent 50 and LCx stent 70 reside within the distal end 30d of LM stent 30. The proximal ends of LAD stent 50 and LCx stent 70 are deformed but not crushed, mangled or widened.

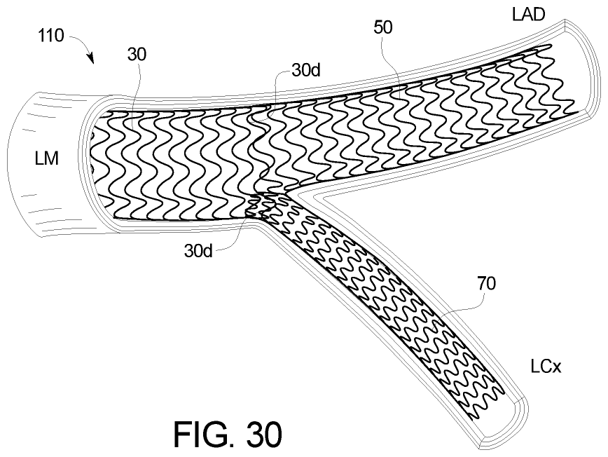

FIG. 30 illustrates that method 110 varies based on the geometry of the anatomy of the patient. In FIG. 30, the diameter of the LCx is smaller in diameter than that of FIGS. 20 to 29, and smaller in diameter than the LAD. The result is that transition section Y may be smaller. In such a case, distal end 30d of LM stent 30 may not have to extend as far, or at all, into transition section. Also, while method 110 illustrates stenting the LAD before the LCx, it is expressly contemplated to reverse that order if the patient's anatomical geometry so dictates. In method 110, the physician is able to tailor the diameter of each stent 30, 50 and 70 to the diameter of the patient's LM, LAD and LCx, respectively. The length of each stent 30, 50 and 70 may be chosen based on the patient's anatomical geometry and/or amount of disease. As discussed above, it is contemplated to package all three stent assemblies 20, 40, 60 (or only two) in a same package. Here, stents 30, 50 and 70 (or only two of same) may each be provided in different size varieties, so that a package having the correct or optimal combination of sizes for stents 30, 50 and 70 (or only two of same) may be selected. Alternatively, stent assemblies 20, 40, 60 are packaged separately, where each package is selected to have a correctly sized stent 30, 50, 70 respectively.

In an alternative embodiment for method 110, it is expressly contemplated after FIG. 24 to instead insert catheter 42 and its associated balloon 46 and stent 50 into the patient's LAD, to insert catheter 62 and its associated balloon 66 and stent 70 into the patient's LCx (in either order), and then to inflate balloons 46 and 66 at least substantially simultaneously to deploy stents 50 and 70 at least substantially simultaneously. Doing so may be desirable so that the interface between the upper portion of proximal end 70up of LCx stent 70 and the lower portion of the proximal end 50lp of LAD stent 50 (FIG. 28) reside roughly halfway within the transition section Y between the LAD and LCx.

As discussed herein, the patient may have additional one or more branches extending off of the LM. In such a case, one of guidewires 90 or 92 may be rewired into the additional branch. Or, a third guidewire may be inserted through deployed LM stent 30. In either case, a fourth stent assembly is inserted through both open ends and within the struts of deployed LM stent 30, and along the rewired or new guidewire only, so as to reside at a desired location within the additional branch. The balloon of the fourth stent assembly is inflated to deploy the fourth stent, and perhaps in combination with inflating one or both balloons 46 and 48 simultaneously. The above procedure is repeated for any additional branches.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1: A left coronary artery (“LCA”) stent system comprising:
a first guidewire having a first diameter;
a second guidewire having a second diameter, which may be the same or different than the first diameter;
a left main (“LM”) stent assembly including (i) a catheter having a saline/contrast solution lumen and a guidewire lumen, the guidewire lumen sized and shaped to accept both the first and second guidewire diameters of the first and second guidewires, such that the first and second guidewires during a procedure extend simultaneously through the catheter and exit a distal tip of the catheter, and (ii) a LM stent sized to be deployed within a patient's LM;
a left anterior descending artery (“LAD”) stent assembly for insertion through a deployed LM stent and along the first guidewire; and
a circumflex artery (“LCx”) stent assembly for insertion through the deployed LM stent and along the second guidewire.

2: The LCA stent system of claim 1, wherein the LM stent assembly, the LAD stent assembly and the LCx stent assembly are provided in a same package.

3: The LCA stent system of claim 1, wherein the LM stent is sized to be deployed within the patient's LM and at least a portion of a transition section in which the LM branches into the LAD and LCx.

4: The LCA stent system of claim 1, wherein a longest cross-sectional length of the saline/contrast solution lumen is commensurate with at least one of the first and second guidewire diameters.

5: The LCA stent system of claim 4, wherein a widest cross-sectional width of the saline/contrast solution lumen is twenty to fifty percent of the longest length of the saline/contrast solution lumen.

6: A left coronary artery (“LCA”) stent system comprising:
a left main (“LM”) stent assembly including a LM stent sized to be deployed within a patient's LM;
a left anterior descending artery (“LAD”) stent assembly including a LAD stent sized differently than the LM stent and to be deployed within the patient's LAD; and
a circumflex artery (“LCx”) stent assembly including a LCx stent sized differently than the LM stent and to be deployed within the patient's LCx,
wherein the LM stent assembly further includes a catheter sized and shaped to accept the diameters of a LAD guidewire and a LCx guidewire, such that the LAD and LCx guidewires during a procedure extend simultaneously through the catheter and exit a distal tip of the catheter.

7: The LCA stent system of claim 6, wherein the LCx stent is sized differently than the LAD stent.

8: The LCA stent system of claim 6, wherein the LM stent assembly, the LAD stent assembly and the LCx stent assembly are provided in a same package.

9: The LCA stent system of claim 6, wherein the LM stent is sized to be deployed within the patient's LM and at least a portion of a transition section in which the LM branches into the LAD and LCx.

10: The LCA stent system of claim 6, wherein the LAD stent and the LCx stent each have a different diameter than the LM stent.

11: The LCA stent system of claim 6, wherein the LAD stent and the LCx stent each have a different length than the LM stent.

12: A left main (“LM”) stent assembly for left coronary artery (“LCA”) cardiovascular treatment comprising:
a first guidewire having a first diameter;
a second guidewire having a second diameter, which may be the same or different than the first diameter;
a catheter sized and shaped to accept both the first and second guidewire diameters of the first and second guidewires, such that the first and second guidewires during the LCA treatment extend simultaneously through the catheter and exit a distal tip of the catheter;
a balloon attached to the catheter and configured to be inflated; and
a stent residing about the balloon and the catheter, the stent sized such that when the balloon is inflated, the stent has a length from 6 mm to 24 mm and a diameter from 4 mm to 6 mm.

13: The LM stent assembly of 12, wherein the catheter includes a guidewire lumen, the guidewire lumen sized and shaped to accept both the first and second guidewire diameters of the first and second guidewires and to allow both the first and second guidewires to extend simultaneously through the catheter.

14: A left coronary artery (“LCA”) stent method comprising:
deploying a left main (“LM”) stent in a patient's LM such that the LM stent has a first open end and a second open end separated by a plurality of stent struts;
inserting at least one of a left anterior descending artery (“LAD”) stent assembly or a circumflex artery (“LCx”) stent assembly through the first opening and within the plurality of stent struts, through the second opening, and into at least one of the patient's LAD or LCx, respectively; and
inflating a balloon of the at least one of the LAD or LCx stent assembly to deploy a stent of the at least one LAD or LCx stent assembly.

15: The LCA stent method of claim 14, which incudes (i) inserting the LAD stent assembly through the first opening, within the plurality of stent struts, through the second opening, and into the patient's LAD, and inflating the balloon of the LAD assembly to deploy the LCx stent before or after (ii) inserting the LCx stent assembly through the first opening, within the plurality of stent struts, through the second opening, and into the patient's LCx, and inflating the balloon of the LCx assembly to deploy the LCx stent.

16: The LCA stent method of claim 15, which further includes removing a stent catheter and the balloon of the stent assembly having the stent deployed in (i) before inserting the stent assembly in (ii).

17: The LCA stent method of claim 15, which further includes leaving in the LAD or the LCx a stent catheter and the balloon of the stent assembly having the stent deployed in (i) while inserting the stent assembly in (ii).

18: The LCA stent method of claim 15, which includes reinflating the balloon inflated in (i) to correct a deformation of the stent deployed in (i) due to the deployment of the stent in (ii).

19: The LCA stent method of claim 14, wherein deploying the LM stent includes inserting a catheter carrying the LM stent over a plurality of guidewires.

20: The LCA stent method of claim 14, wherein the LM stent is sized to be deployed (i) in the LM only or (ii) within the patient's LM and at least a portion of a transition section where the LM branches into the LAD and LCx.

21: The LCA stent method of claim 14, which includes deploying the LM stent and the at least one of the LAD or LCx stent such that a distal end of the LM stent extends over a proximal end of the at least one of the LAD or LCx stent.

22: The LCA stent method of claim 14, which includes deploying both the LAD and LCx stents at least substantially simultaneously.

23: The LCA stent system of claim 1, wherein at least two of the LM stent assembly, the LAD stent assembly and the LCx stent assembly are provided in different packages.

24: The LCA stent system of claim 1, wherein at least one of the first guidewire diameter or the second guidewire diameter is circular.

25: The LCA stent system of claim 6, wherein at least two of the LM stent assembly, the LAD stent assembly and the LCx stent assembly are provided in different packages.

26: The LCA stent system of claim 6, wherein the catheter includes a guidewire lumen, the guidewire lumen sized and shaped to accept the diameters of LAD and LCx guidewires and to allow both the LAD and LCx guidewires to extend simultaneously through the catheter.

27: The LCA stent system of claim 26, wherein at least one of the LAD guidewire diameter or the LCx guidewire diameter is circular.

28: The LCA stent system of claim 1, wherein at least one of (i) the distal tip is a radiopaque tip or (ii) the distal tip is a same distal tip for the first and second guidewires.

29: The LCA stent system of claim 6, wherein at least one of (i) the distal tip is a radiopaque tip or (ii) the distal tip is a same distal tip for the LAD and LCx guidewires.

30: The LM stent assembly of claim 12, wherein at least one of (i) the distal tip is a radiopaque tip or (ii) the distal tip is a same distal tip for the first and second guidewires.
